(19) United States
(12) Patent Application Publication
(10) Pub. No.: US 2025/0061975 A1
(43) Pub. Date: Feb. 20, 2025

(54) SYSTEM AND METHOD FOR DETERMINING GLYCAN TOPOLOGY USING DE NOVO GLYCAN TOPOLOGY RECONSTRUCTION TECHNIQUES

(71) Applicants: BRANDEIS UNIVERSITY, Waltham, MA (US); TRUSTEES OF BOSTON UNIVERSITY, Boston, MA (US)

(72) Inventors: Pengyu HONG, Waltham, MA (US); Cheng LIN, Lynnfield, MA (US)

(21) Appl. No.: 18/724,160

(22) PCT Filed: Dec. 29, 2022
(86) PCT No.: PCT/US2022/082587

§ 371 Date: Jun. 25, 2024

Related U.S. Application Data
(60) Provisional application No. 63/294,681, filed on Dec. 29, 2021.

(51) Int. Cl. G16C 20/20; H01J 49/00

(52) U.S. Cl. CPC G16C 20/20; H01J 49/0036

(57) ABSTRACT

Provided herein are systems and methods for determining the topology of a molecule from mass spectrometry data.

### CROSS-REFERENCE TO RELATED APPLICATIONS

This application claims priority to U.S. Provisional Application No. 63/294,681 filed on Dec. 29, 2021, the contents of which is incorporated by reference in its entirety.

### GOVERNMENT SUPPORT

This invention was made with government support under GM134210, and GM 132675 awarded by the National Institutes of Health, and 1920147 awarded by the National Science Foundation. The government has certain rights in the invention.

### BACKGROUND

Glycosylation is a highly regulated process, in which one or more glycans (or oligosaccharides) is added to a protein or lipid and remodeled after attachment, with both stages being under the control of specific enzymes. It plays an essential role in various biological processes [1-3], such as protein folding, immunological response, signal transduction, cell adhesion, and so on. Previous studies show that the change in glycosylation patterns is frequently associated with pathological characteristics [4, 5]. Proper glycosylation is essential to achieve the required solubility, stability and efficacy of many biopharmaceuticals [6, 7]. Therefore, glycan structural analysis is critical for understanding the multiple biological roles of glycosylation. Tandem mass spectrometry (MS/MS) is a widely used tool for elucidating the detailed structures of glycans [8, 9]; these consist of monosaccharides linked by glycosidic bonds. The larger glycans can be multiply branched and thus have tree-like structures. In an MS/MS experiment, a glycan may be cleaved into fragments, forming a mass/charge spectrum composed of structural components that have been designated as glycosidic (B-, C-, Y-, Z-), cross-ring (A-, X-) and internal fragments [10]. Accurate deduction of the glycan topology, i.e. its two-dimensional sequence, requires cleavages of every single glycosidic bond in an MS/MS experiment. However, MS/MS spectra are typically noisy and some sequence ions (glycosidic fragments) may be missing. In addition, the number of potential topologies (i.e., the search space) is huge, even for a moderate-sized glycan. Therefore, it is challenging to reconstruct the fully defined glycan structure from an MS/MS spectrum.

Database searching approaches [11-14] retrieve glycan topology candidates by matching an experimentally acquired MS/MS spectrum with those of known glycans in their databases. The performance of this type of approach highly depends on the coverage of the databases, as well as the quality of MS/MS data in the databases, which unfortunately are generally incomplete. Brute-force search methods (e.g., [15]) compare an experimental MS/MS spectrum to those of all possible theoretical structures, but they can work only for small glycans because the number of possible structures increases exponentially with respect to the glycan size. Although biosynthetic rules can be added to speed up topology searches by brute-force methods [16, 17], our knowledge of the glycan biosynthetic rules remains limited. Several approaches grow topology candidates by exploring the relationships between peaks (i.e., mass differences corresponding to known fragments) [18-23]. To make computation feasible, it is natural to limit the size of intermediate results by only keeping a subset of high-scoring sub-topologies [18, 19] or applying a mass tolerance threshold [20, 22]. Different from other approaches that use manually designed functions to score structure candidates, machine learning-based techniques were developed to establish better scoring functions from experimental data [21, 22]. However, neither a score nor a ranking of a topology candidate indicates its statistical significance. In addition, the speeds of the aforementioned approaches are still not fast enough for real-time inference. Real-time execution is needed for dynamic selection of the right fragments to achieve efficient and effective MS³ analysis.

Currently, there is a need for a topology reconstruction technique that speeds up reconstruction of candidate topologies with reduced computational complexity, and through use of a method that does not rely on a database of known structures.

### SUMMARY

The present disclosure overcomes the aforementioned drawbacks by providing systems and methods for de novo reconstruction of molecule topologies from mass spectrometry data. The provided systems and methods offer functionality to calculate p-values of reconstructed topologies. The provided systems and methods allow for the determination of monomer subunit compositions for molecules satisfying any given precursor mass, within defined mass measurement accuracy limits, which can then be used to constrain the search space of potential topologies. The mapping from masses to monomer subunit compositions can be precomputed. A theoretical spectrum can be pre-computed for each monomer subunit composition to include the theoretical fragment ions of all topology candidates that satisfy a user-specified monomer subunit composition constraint. Given an experimental MS/MS spectrum, the provided systems and methods retrieve monomer subunit compositions and their theoretical spectra, which are within the mass accuracy of the experimental precursor mass. The retrieved theoretical spectra are then filtered by the experimental spectrum before being used for reconstructing topology candidates. The number of peaks in such a filtered theoretical spectrum is substantially smaller than that in the experimental spectrum. Hence, it takes considerably shorter time to reconstruct topologies from a filtered theoretical spectrum.

In one aspect, the present disclosure provides a method for determining a topology for a molecule. The method includes acquiring a mass spectrum of a molecule, where the mass spectrum includes mass spectrum peaks corresponding to a precursor ion and fragment ions, where the precursor ion corresponds to an ionized product of the molecule and the fragment ions correspond to dissociated products of the molecule. The method further includes matching mass spectrum peaks in the mass spectrum with theoretical mass spectrum peaks of a theoretical spectra of the molecule, and producing a filtered mass spectrum of the molecule by removing unmatched mass spectrum peaks from the mass spectrum. The method further includes identifying at least a portion of the fragment ions in the filtered mass spectrum as corresponding to one or more monomer subunit ion of the precursor ion, wherein the one or more monomer subunit ion is identified by appending one or more of the fragment ions to an inferable constituent to produce a topology building block, and storing the topology building block in a candidate pool as corresponding to one or more of the monomer subunit ion if the combined mass of the inferable constituent and one or more of the fragment ions satisfy a first user-defined mass tolerance. The method further includes reconstructing one or more candidate topology of the precursor ion by combining a plurality of the topology building blocks that satisfy a second user-defined mass tolerance for the precursor ion.

In another aspect, the present disclosure provides a mass spectrometry unit that comprises an inlet port configured to receive a sample that includes a macromolecule comprising monomer subunits, and an ion source configured to ionize the sample to produce a precursor ion, the precursor ion having a first mass-to-charge ratio. The mass spectrometry unit also includes a mass analyzer configured to dissociate a portion of the precursor ion to produce fragment ions, where the mass analyzer configured to separate a fraction of the precursor ion and the fragment ions. A detector may also be configured to produce detection signals corresponding to the fraction of the precursor ion and the fragment ions. The mass spectrometry unit may further include a controller configured to receive the detection signals, the controller programmed to: acquire a mass spectrum of the molecule, the mass spectrum including mass spectrum peaks corresponding to a precursor ion and fragment ions, wherein the precursor ion corresponds to an ionized product of the molecule and the fragment ions correspond to dissociated products of the molecule. The controller is further programmed to match mass spectrum peaks in the mass spectrum with theoretical mass spectrum peaks from a theoretical spectra of the molecule, and produce a filtered mass spectrum of the molecule by removing unmatched mass spectrum peaks from the mass spectrum. The controller is further programmed to identify at least a portion of the fragment ions in the filtered mass spectrum as corresponding to one or more monomer subunit ion of the precursor ion, wherein the one or more monomer subunit ion is identified by appending one or more of the fragment ions to an inferable constituent to produce a topology building block, and storing the topology building block in a candidate pool as corresponding to one or more of the monomer subunit ion if the combined mass of the inferable constituent and one or more of the fragment ions satisfy a first user-defined mass tolerance. The controller is further programmed to reconstruct one or more candidate topology of the precursor ion by combining a plurality of the topology building blocks that satisfy a second user-defined mass tolerance for the precursor ion.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1A is an illustration of a glycan fragmentation nomenclature system for use in accordance with the present disclosure.

FIG. 1B is a linear representation, a two-dimensional representation, and a graphic representation of a glycan structure for use in accordance with the present disclosure.

FIG. 2 is a graphical illustration of an example method for determining a topology of a molecule in accordance with one aspect of the present disclosure.

FIG. 3 is a block diagram illustrating an example of a computer system that can implement some aspects of the present disclosure.

FIG. 4 is a block diagram of a mass spectrometry unit that can implement some aspects of the present disclosure.

FIG. 5 is a graphical illustration of an example method for determining a topology of a molecule in accordance with one aspect of the present disclosure.

FIG. 6 is a distribution of the number of monosaccharaide compositions with respect to the protonated m/z of the precursor ions, wherein each dot indicates the number of monosaccharide compositions of one mass.

FIG. 7 is a graph comparing the speeds of Glyco DeNovo and GlycoDeNovo2, where each dot represents one experimental spectrum.

FIG. 8 is a graph comparing the number of peaks used in topology reconstruction, where each dot represents one experimental spectrum.

### DETAILED DESCRIPTION

Described herein are systems and methods for determining a topology or molecular formula of a molecule using mass spectrometry data. Suitable molecules for use with the systems and methods presented herein may include macromolecules and small molecules. As used herein, a macromolecule may comprise any repeatable unit (e.g., monomer subunit) or pairs of units that may be coupled together to produce the macromolecule. Exemplary molecules of the present disclosure may include natural and synthetic macromolecules. Non-limiting examples of natural macromolecules include, but are not limited to carbohydrates or glycans (e.g., composed of monosaccharides), nucleic acids (e.g., composed of nucleotides), proteins and/or peptides (e.g., composed of amino acids), lipids (e.g., composed of fatty acids), derivatives and mixtures thereof. Suitable synthetic macromolecules include, but are not limited to, one or more monomer subunit selected from ethylene, propylene, styrene, tetrafluoroethylene, vinyl chloride, derivatives and mixtures thereof.

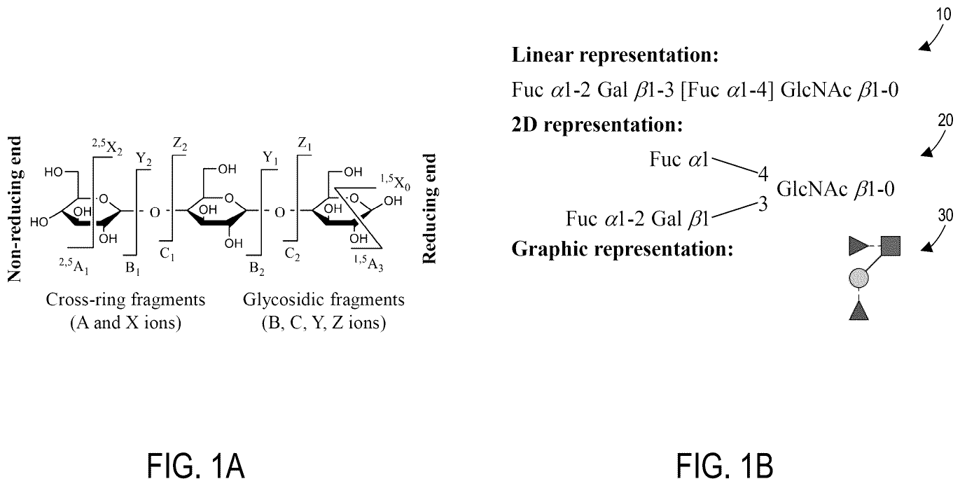

Owing to the structure complexity of glycans, the technology for determining glycan structure from experimental data has lagged behind those for other classes of biological macromolecules. In one embodiment, the methods described herein can accurately and efficiently determine the topology or molecular formula for glycans using experimental data. Referring to FIGS. 1A-B, a non-limiting example of a glycan is provided to illustrate dissociation patterns of glycans during mass spectroscopy experiments. As shown in FIG. 1A, a single glycosidic cleavage during a mass spectroscopy experiment produces monomer subunit ions, such as B-, C-, Y-, and Z-ions, whereas cross-ring cleavages generate fragment ions, such as, A- and X-ions. Internal fragment ions, or fragment ions with loss of multiple branches may also be formed by two or more glycosidic and/or cross-ring cleavages. In some aspects, the methods presented herein group fragment ions, such as A- and X-ions, and internal fragment ions into a category termed O-ions (i.e., Other ions). The monomer subunit glycosidic fragments are important for topology deduction. Since a Y ion differs in mass from its related Z-ion by that of a water molecule, as does a B ion from its related C-ion, C- and Z-ions provide redundant information to B- and Y-ions. A- and X-ions are useful for deciphering the branching pattern and linkages, as well as for ranking the candidate topologies. The topology of a glycan can be represented as a tree with nodes representing monosaccharide residues and edges representing glycosidic linkages. For example, FIG. 1B provides an illustration of a linear representation 10 of a glycan, a two-dimensional representation 20 of a glycan, and a graphic representation of a glycan 30.

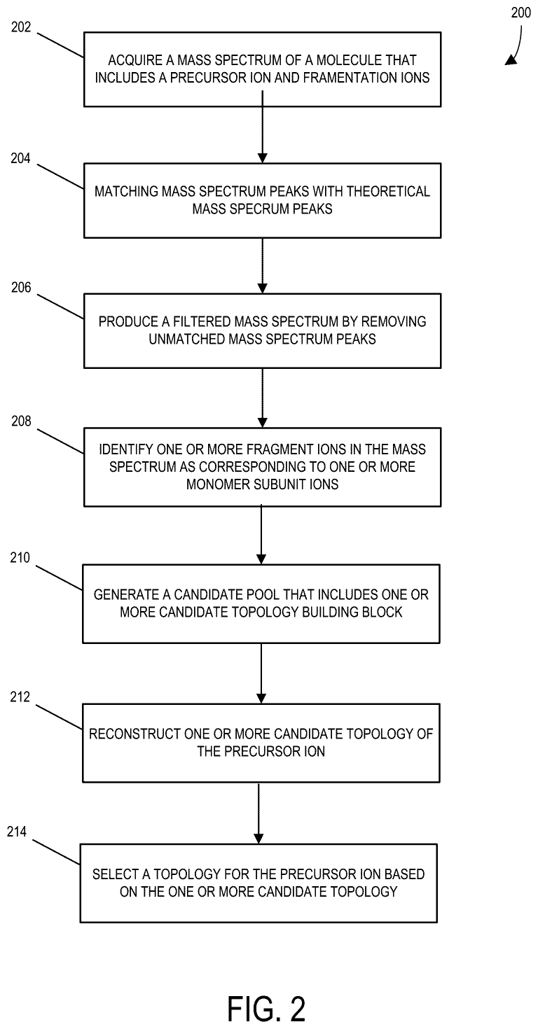

Referring to FIG. 2, a flowchart is provided as setting forth the steps of an example method 200 for determining a topology of a molecule in accordance with the present disclosure. The method 200 may also be referred to throughout the disclosure as “GlycoDeNovo2.” The method 200 includes acquiring a mass spectrum of a molecule having mass spectrum peaks corresponding to a precursor ion and fragment ions, as indicated at step 202. In some aspects, the precursor ion corresponds to an ionized product of the molecule and the fragment ions correspond to dissociated products of the molecule. As used herein, “acquiring” the mass spectrum may include providing previously acquired data to a computer system from a memory or other data storage device, or may including acquiring a mass spectrum using a mass spectrometry unit and communicating the acquired data to a computer system, which may form a part of the mass spectrometry unit.

In some aspects, the method 200 includes preprocessing the mass spectrum of the molecule. Preprocessing the mass spectrum may include, but is not limited to protonating all the peaks in the spectrum, performing a baseline correction, spectral alignment of profiles, normalization, peak preserving noise reduction, peak finding with wavelet denoising, binning through peak coalescing and combinations thereof. Further, it is common that some fragment ions are unobservable in the experimental spectrum due to secondary fragmentations or lack of charge carriers. In some aspects, the method 200 includes preprocessing the mass spectrum to identify and add in computed complementary peaks missing from the mass spectrum. For example, in theory, when a glycan is cleaved only once, two complementary ions should appear. Hence, missing peaks can be recovered from their complementary peaks. For example, B-/C-/A-ions can be recovered from Y-/Z-/X-ions, respectively, and vice versa. Since the precursor ion is known, one can calculate the complementary peak of each experimentally observed peak and add a computed peak to the spectrum if it is missing in the original spectrum. Then preprocessing may include iteratively merging peaks that are within 0.001 Dalton starting from the closest pair of peaks.

In some aspects, the method 200 further includes matching mass spectrum peaks in the mass spectrum with theoretical mass spectrum peaks of a theoretical spectrum of the molecule, as indicated in step 204. The method 200 further includes producing a filtered mass spectrum of the molecule by removing unmatched mass spectrum peaks from the mass spectrum, as indicated by step 206.

In some aspects, the theoretical spectrum may be obtained from a precomputed mass-to-composition database DB_{M2C}. The mass-to-composition database DB_{M2C} may be indexed by precursor masses and store a portion or all possible monomer subunit ion compositions of the molecule with precursor masses smaller than a predefined threshold Mₘₐₓ. In some aspects, DB_{M2C} also stores the theoretical spectra corresponding to each monomer subunit ion. The DB_{M2C} may be precomputed and stored in a memory or other data storage device. Alternatively, the DB_{M2C} may be produced. In some aspects, the method 200 includes producing the theoretical spectrum of the molecule by deriving monomer subunit ions in a recursive way. For example, in some aspects, the method 200 starts with an empty composition and calls itself recursively to expand the composition by adding one monomer subunit ion each time to meet a mass accuracy constraint of the molecule. The method 200 may further include calculating the theoretical spectrum of the molecule as a union of all protonated monomer subunit ions from a portion or all possible monomer subunit compositions that satisfy the molecule constraint.

In one non-limiting example, the theoretical spectrum of the molecule may be produced using algorithms dubbed, “Mass2Composition” and “Composition2Spectrum.” Mass2Composition derives the monomer subunit compositions in a recursive way and Composition2Spectrum calculates the theoretical spectrum of the molecule.

In one non-limiting example, Mass2Composition may be represented by:

Algorithm 1: Mass2Composition (C = [c₁, c₂, ..., cₖ], M, d)

```
/* Input: C is the input monosaccharide composition. The monosaccharides are ordered
from the lightest to the heaviest. M is the corresponding mass of the input
monosaccharide composition, and d is the derivatization method used to produce the
MS/MS spectrum. Set C = [0, ..., 0] and M = 0 when calling Mass2Composition the first
time.*/
for all mᵢ ∈ monosaccharide class set G do
  Let C_{new} = [c₁, ..., cᵢ₊₁, ..., cₖ]
  Let M_{new} = M + f(d, mᵢ), where the function f decides the mass increase due to adding a
    monosaccharide mi to C. The mass increase depends on the derivatization d and
    the mass loss caused by forming a new glycosidic bond.
  if M_{new} > Mₘₐₓ or [M_{new}, C_{new}, d] ∈ DB_{M2C} then
    return
  else
    /* Calculate the theoretical spectrum S of C_{new} */
    S = Composition2Spectrum (C_{new}, d)
    Add [M_{new}, C_{new}, d, S] to DB_{M2C}.
    Mass2 Composition (C_{new}, M_{new}, d)
  end
end
```

In one non-limiting example, Composition2Spectrum may be represented by:

Algorithm 2: Composition2Spectrum (C = [c₁, c₂, ..., cₖ], d)

```
/* Input: C is the input monosaccharide composition, and d is the derivatization method
used to produce the MS/MS spectrum. Output: The theoretical spectrum S of C. */
Initialize the theoretical spectrum S = Ø
Let N= be the total number of monosaccharides in C.
for n = 1 to N do
  for all τ ∈ unique (choose n monosaccharides from C)
    Let τ be the monosaccharide composition of a non-reducing-end fragment
    Generate the corresponding protonated B-, C-, Y-, and Z-ions as B_{τ}, C_{τ}, Y_{τ}, and Z_{τ},
      respectively.
    Add B_{τ}, C_{τ}, Y_{τ}, and Z_{τ} to S.
  end
end
return S.
```

In some aspects, the method 200 includes identifying at least a portion of the fragment ions in the filtered mass spectrum as corresponding to one or more monomer subunit ion of the precursor ion, as indicated in step 208. Identifying the fragment ions as monomer subunit ions may include appending one or more of the fragment ions to an inferable constituent to produce a candidate topology building block. As indicated in step 210, the candidate topology building block may then be stored in a candidate pool as corresponding to one or more of the monomer subunit ions if the combined mass (or mass-to-charge ratio) of the inferable constituent and the one or more fragment ions satisfies a user-defined mass tolerance. For example, satisfying the user-defined mass tolerance may be achieved if the combined mass-to-charge ratio of the inferable constituent and the one or more fragment ion falls within a specified range around a predicated combined mass of the inferable constituent and the one or more fragment ion. In one non-limiting example, the user-defined mass tolerance may be 0.02 Da or less (or the m/z equivalent). In other aspects, the user-defined mass tolerance may be 0.005 Da or less (or the m/z equivalent). In some aspects, the user-defined mass tolerance ranges between 0.005 and 0.02 Da (or the m/z equivalent).

In some aspects, the candidate topology building block is produced by first identifying lighter fragment ions in the filtered mass spectrum as corresponding to one or more monomer subunit ion, and proceeds by searching for some or all allowable combinations of fragment ions in the candidate pool that can be appended to an inferable constituent to obtain the candidate topology building block with a mass within the first user-defined mass tolerance. In one non-limiting example, steps 208-210 may include identifying fragment peaks as corresponding to B or C glycosidic ions (e.g., monomer subunit ions) of a glycan ion (e.g., precursor ion) by using interpretations of preceding peaks. In each iteration, the method 200 interprets some or all of the fragment ion peaks as corresponding to B or C glycosidic ions by attaching up to four branches to a monosaccharide (e.g., inferable constituent), wherein the branches are interpretations of fragment ion peaks that are lighter than the one being interpreted. In some aspects, the monomer subunit ions correspond to a non-reducing end of a glycosidic fragment. The candidate topology building blocks may be represented in graphical form. For example, in some aspects, steps 208-210 include generating an interpretation-graph that includes nodes and edges to respectively represent fragment peaks and how a fragment peak can be interpreted as a monomer subunit ion by using interpretations of preceding peaks.

In some aspects, the method 200 includes reconstructing one or more candidate topology of the precursor ion by combining multiple candidate topology building blocks to satisfy a second user-defined mass tolerance for the precursor ion, as indicated in step 212. In some aspects, the method 200 includes reconstructing all the possible candidate topologies for the precursor ion. In one non-limiting example, the user-defined mass tolerance may be 0.02 Da or less (or the m/z equivalent). In other aspects, the user-defined mass tolerance may be 0.005 Da or less (or the m/z equivalent). In some aspects, the user-defined mass tolerance ranges between 0.005 and 0.02 Da (or the m/z equivalent).

The method 200 may also include selecting a topology for the precursor ion by ranking the one or more candidate topology based on a candidate topology score, and selecting the candidate topology having the highest candidate topology score, as indicated by step 214. In some aspects, selecting the topology for the precursor ion includes applying a machine-learning technique to generate a candidate topology score. The candidate topology score may be based on the likelihood that the fragment ions in the mass spectrum correspond to the one or more monomer subunit ion identified in the candidate pool. The candidate with the highest candidate topology may then be selected as the topology for the precursor ion. In one non-limiting example, the candidate topology score may include defining a mass difference window in the mass spectrum that includes one or more of the fragment ions in the mass spectrum, and expressing the fragment ions as an array of contextual features to determine if the fragment ions in the mass difference window correspond to a monomer subunit ion. A positive value may then be assigned to mass spectrum peaks that contain the highest likelihood of corresponding to a monomer subunit ion based on the array of contextual features, and a negative value may be assigned to mass spectrum peaks that contain the lowest likelihood of corresponding to a monomer subunit ion based on the array of contextual features.

In one non-limiting example, steps 208-212 may be performed using an algorithm dubbed, “PeakInterpreter2.” In some aspects, PeakInterpreter2 builds an interpretation-graph that specifies how to interpret each peak using the topologies of other peaks with lighter masses. In some aspects, PeakInterpreter2 takes the interpretation-graph and reconstructs all candidate topologies of the precursor ion that satisfy the user-defined mass accuracy constraint. The algorithms are provided in detail below, along with symbols and data structures used. However, these algorithms are provided for illustration only, and are not intended to limit the disclosure. In one non-limiting example, PeakInterpreter2 may be represented by:

Algorithm 3: PeakInterpreter2 (C = [c₁, c₂, ..., cₖ], Sₑₓₚₑᵣᵢₘₑₙₜ)

```
/* Input: C is the monosaccharide composition. Sexperiment is the preprocessed
experimental spectrum. Output: Topology reconstruction results. */
Retrieve the theoretical spectrum S_{theory} of C from DB_{M2C}.
Obtain S_{filtered} by removing peaks in S_{theory} that are not matched in Sₑₓₚₑᵣᵢₘₑₙₜ.
Initialize the topology candidate pool T = Ø.
for each peak n in S_{filtered} from the lightest to the heaviest do
  Initialize a candidate t_{new}:
  Set the mass t_{new}.mass = the mass of n.
  Set the topology super sets t_{new}.TSS = Ø.
  for all possible combinations of up to 4 candidates tₐ, t_{b}, t_{c}, t_{d} ∈ T do
    Find a monosaccharide m so that the topologies (using m as the root and tₐ, t_{b}, t_{c}, t_{d}
      as branches) satisfy the composition constraint C and match the mass of n.
    If such m exists, create a topology set aTS and set aTS.root = m and aTS.branches =
      [tₐ, t_{b}, t_{c}, tₐ]. Add aTS to tnew. TSS.
  end
  if t_{new}.TSS == Ø then
    Add t_{new} to T.
  end
end
```

PeakInterpreter2 may allow candidate topologies to have up to 4 branches at each branching point. In some aspects, this constraint may be lowered to increase computation speed, or it may be increased for some monomer subunit ions. PeakInterpreter2 maintains a candidate pool where each candidate topology building block serves as a potential building block for interpreting a heavier peak. PeakInterpreter2 starts from the lightest peak and tries to interpret some or all of the mass spectrum peaks as a monomer subunit ion (e.g., B ion and C ion) or the precursor ion by searching for all allowable combinations of fragment ions in the candidate pool S that can be appended to a root or inferable constituent (e.g., monosaccharide) g to obtain a candidate set or pool with a mass within the accuracy range specified by τ. In some aspects, the mass difference & depends on the ion type and macromolecule derivation method deployed, (i.e., permethylation). The intensities of the non-precursor peaks may be interpretable by PeakInterpreter2 to normalize the intensities of all peaks into z-scores.

After obtaining the interpretation-graph, the candidate set object of the precursor ion is reconstructed into legal candidate topologies (e.g., fall within a user-defined mass tolerance). PeakInterpreter2 creates legal topologies of r, which are rooted and satisfy the mass accuracy constraint. The branches are linked by their alphabetic order so that isomorphic topologies can be effectively detected and removed.

In some embodiments, the method 200 further includes selecting a topology for the precursor ion by ranking one or more candidate topology based on a candidate topology score. In some aspects, the candidate topology score is based on identifying the probability that the fragment ions correspond to a B ion glycosidic fragment or a C ion glycosidic fragment. An algorithm dubbed “IonClassifer” may be used to distinguish different types of fragment ions and score candidate topologies. In some aspects, IonClassifier takes a peak and its context, currently defined as the neighboring peaks within a pre-determined mass-difference window (e.g., 105 Da), and classifies the peak as +1 (i.e., a B-or C-ion) or −1 (i.e., a non-B or C ion). The neighboring peaks can be expressed as an array of contextual features (e.g., mass shifts) from the peak of interest. The final score of a candidate topology is calculated by summing up the IonClassifier values of its supporting peaks.

In some aspects, IonClassifier may be trained by boosting the decision tree classifier on experimental tandem mass spectra of a set of known macromolecules. For each macromolecule standard, a computer system or mass spectrometry unit can match its theoretical spectrum to the experimental spectrum to collect the observed context of each theoretical peak found in the experimental spectrum. In one non-limiting example, the computer system or mass spectrometry unit can then group the supporting peaks of candidates into true B-ions, true C-ions, true Y-ions, true Z-ions, and O-ions, and trained IonClassifier to distinguish true B-ions and true C-ions from Y-, Z-, and O-ions. If a supporting peak is interpreted by PeakInterpreter2 as a B ion, it will be validated by the B-ion classifier of IonClassifier. Similarly, if a supporting peak is interpreted by PeakInterpreter2 as a C-ion, it will be validated by the C-ion classifier of IonClassifier.

In some embodiments, the method 200 includes generating an empirical p-value for the candidate topology score of the one or more candidate topology. In some aspects, generating the empirical p-value includes sampling theoretical topologies from a precomputed composition-to-topology database DB_{C2T} and using the empirical distribution to generate the empirical p-value of the one or more candidate topology. The composition-to-topology database DB_{C2T} allows one to retrieve all topologies using a monomer subunit composition query. DB_{C2T} organizes topologies and their sub-topologies into topology sets and topology super sets. A topology super set contains all topologies (or sub-topologies) of the same monosaccharide composition, which are organized in topology sets. A topology set contains topologies (or sub-topologies) that have the same monomer subunit composition, are rooted at the same monomer subunit, and share the same branching pattern at its root. A branching pattern specifies the number of branches of all topologies (or sub-topologies) in this topology set and the monomer subunit composition of each branch (i.e., each branch contains a set of sub-topologies in a topology super set). The topology sets and topology super sets are stored in two cross-referred databases, DB_{C2TS} and DB_{C2TSS}, respectively. DB_{C2TS} and DB_{C2TSS} together effectively organize all topologies and sub-topologies in a directed acyclic graph (DAG), which is similar to the interpretation-graph. Each node in this DAG is either a topology set or a topology super set. A comprehensive DB_{C2T} can be pre-computed by traversing this DAG and be used later in calculating the p-value of a topology candidate. It is also indexed by the masses of topologies and stores the theoretical spectrum of each topology. For very large glycans, the number of possible topologies can be too large to pre-compute and store offline. For the purpose of computing empirical p-values, we can instead sample the DAG to obtain the desired number of topologies.

In some aspects, the method 200 includes generating DB_{C2TS} and DB_{C2TSS}. DB_{C2TS} and DB_{C2TSS} may be generated using two algorithms, Composition2TSS (Algorithm 4) and CreateRootedTSS (Algorithm 5). Composition2TSS takes a monomer subunit composition C=[c₁, c₂, . . . , cₖ] as input and recursively reconstructs and saves typologies (or sub-topologies) satisfying this composition. The algorithm iterates through available monomers in C. Each time, it picks a monomer, say mᵢ, as a root, and then calls the algorithm CreateRootedTSS (Algorithm 4) with the remaining composition to create all topologies (or sub-topologies) rooted at mi.

In one non-limiting example, Composition2TSS may be represented by:

Algorithm 4: Composition2TSS (C = [c₁, c₂, ..., cₖ])

```
/* Inputs: C is the input monosaccharide composition. This function creates all
topologies satisfying the input composition constraint and return them in a topology
super set object aTSS.Save aTSS in DB_{C2TSS} and index it by C. * /
if C is not empty then
  if C ∈ DB_{C2TSS} then
    Retrieve the topology super set aTSS of C from DB_{C2TSS}.
  else
    Create a new topology super set aTSS.
    for ∀cᵢ > 0 do
      C_{new} = [c₁, ..., cᵢ₋₁, ..., cₖ]
      rtss = CreateRootedTSS(mᵢ, C_{new}), where mi is the i-th monosaccharide to be
      used as the root.
      Add the topology sets in rtss to aTSS.
    end
  end
  Save aTSS to DB_{C2TSS} and index it by C.
  return aTSS.
end
return null.
```

In one non-limiting example, CreateRootedTSS may be represented by:

Algorithm 5: CreateRootedTSS (root, C = [c₁, c₂, ..., cₖ])

```
/* Input: root is the monosaccharide to be used as the root in all topologies whose
branches have a total composition as C. Output: a topology super set aTSS that contains
all the topologies that are rooted at root and satisfy the composition constraint. */
Create a new topology super set aTSS.
if C == Ø then
  if root, Ø, Ø, Ø, Ø ∈ DB_{C2TS} then
    Retrieve the topology set aTS of root, Ø, Ø, Ø, Ø from DB_{C2TS}.
  else
    Create a new topology set aTS and set aTS.root = root.
    Add aTS to DB_{C2TS} using root, Ø, Ø, Ø, Ø as the key.
  end
  Add aTS to aTSS.
else
  for all up-to-4 partitions of C as C₁, C₂, C₃, C₄ do
  /* Cᵢ specifies the monosaccharide composition of the i- th branch */
    if root, C₁, C₂, C₃, C₄ ∈ DB_{C2TS} then
      Retrieve the topology set aTS of root, C₁, C₂, C₃, C₄ from DB_{C2TS}.
    else
      Create a new topology set aTS aTS.root = root.
      aTS.branches[1] = Composition2TSS (C₁)
      aTS.branches[2] = Composition2TSS (C₂)
      aTS.branches[3] = Composition2TSS (C₃)
      aTS.branches[4] = Composition2TSS (C₄)
      Add aTS to DB_{C2TS} using root, C₁, C₂, C₃, C₄ as the key.
    end
    Add aTS to aTSS.
  end
end
return aTSS.
```

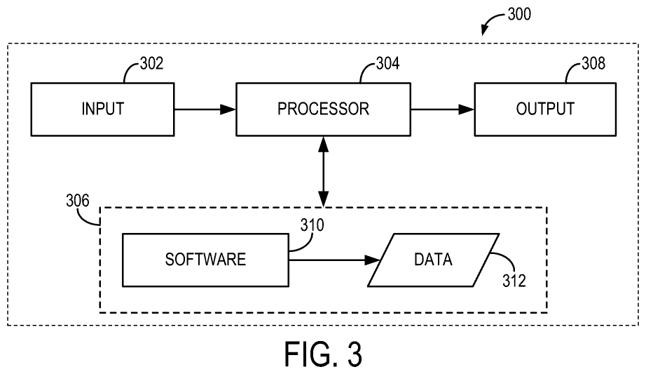

Referring now to FIG. 3, a block diagram of an example of a computer system 300 that can be used to implement the methods described herein and, specifically, determine a topology or molecular formula for a molecule using mass spectrometry data. The computer system 300 generally includes an input 302, at least one hardware processor 304, a memory 306, and an output 308. Thus, the computer system 300 is generally implemented with a hardware processor 304 and a memory. In some embodiments, the computer system 300 can be implemented, in some examples, by a workstation, a notebook computer, a tablet device, a mobile device, a multimedia device, a network server, a mainframe, one or more controllers, one or more microcontrollers, or any other general-purpose or application-specific computing device.

The computer system 300 may operate autonomously or semi-autonomously, or may read executable software instructions from the memory 306 or a computer-readable medium (e.g., a hard drive, a CD-ROM, flash memory), or may receive instructions via the input 302 from a user, or any another source logically connected to a computer or device, such as another networked computer, server. The input 302 may take any shape or form, as desired, for operation of the computer system 300, including the ability for selecting, entering, or otherwise specifying parameters consistent with operating the computer system 300.

In general, the computer system 300 is programmed or otherwise configured to implement the methods and algorithms in the present disclosure, such as those described with reference to FIG. 2. For instance, the computer system 300 can be programmed to generate a topology for a molecule based on experimental mass spectroscopy data. In some aspects, the computer system 300 may be programmed to access acquired data from a mass spectrometry unit, such as mass spectroscopy data that includes mass spectrum peaks corresponding to a precursor ion and fragment ions. Alternatively, the mass spectrum may be provided to the computer system 300 by acquiring the data using a mass spectrometry unit and communicating the acquired data to the computer system 300, which may be part of the mass spectrometry unit.

The computer system 300 may be further programmed to process the mass spectrum to generate a topology for the molecule of interest. The computer system 300 may identify at least a portion of the fragment ions in the mass spectrum as corresponding to one or more monomer subunit ion of the precursor ion, and the one or more identified monomer subunit ion may be used to generate a candidate pool containing one or more candidate topology building block. From the one or more candidate topology building block, the computer system 300 may reconstruct a candidate topology of the precursor ion that satisfy a user-defined mass tolerance for the precursor ion.

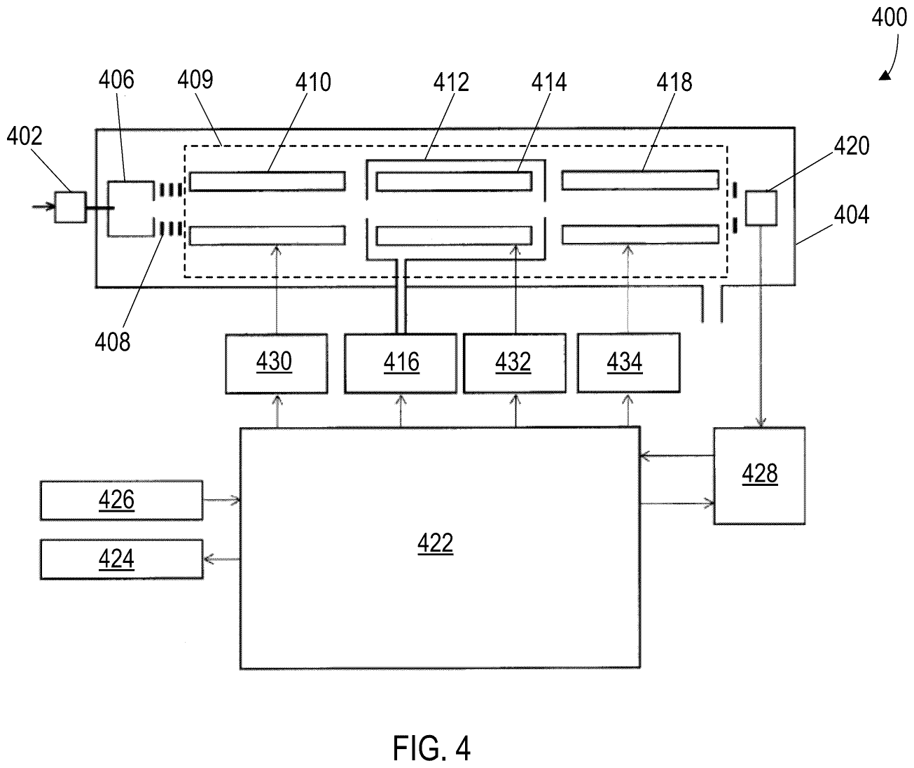

The input 302 may take any suitable shape or form, as desired, for operation of the computer system 300, including the ability for selecting, entering, or otherwise specifying parameters consistent with performing tasks, processing data, or operating the computer system 300. In some aspects, the input 302 may be configured to receive data, such as data acquired with a mass spectrometry unit, such as the system described in FIG. 4. Such data may be processed as described above to generate a topology for the molecule of interest. In addition, the input 302 may also be configured to receive any other data or information considered useful for determining the topology of the molecule using the methods described above.

Among the processing tasks for operating the computer system 300, the one or more hardware processors 304 may also be configured to carry out a number of post-processing steps on data received by way of the input 302. For example, the processor 304 may be configured to generate a topology for the molecule using experimental mass spectrometry data. The processor 304 may be configured to implement the same or similar method tasks as described in FIG. 2.

The memory 306 may contain software 310 and data 312, such as data acquire with a mass spectrometry unit, and may be configured for storage and retrieval of processed information, instructions, and data to be processed by the one or more hardware processors 304. In some aspects, the software may contain instructions directed to processing the input mass spectrum or mass spectroscopy data to be processed by the one or more hardware processors 304. In some aspects, the software 310 may contain instructions directed to processing the mass spectroscopy data or mass spectrum in order to generate a topology of the molecule, as described in FIG. 2. The software may also contain instructions directed to generating a linear representation, a 2D representation, or graphical representation of the topology of the molecule. In some aspects, the software may also contain instructions directed to generating the interpretation-graph, as described in FIG. 2.

Referring now to FIG. 4, an example of a mass spectrometry unit 400 that can implement the methods described here is illustrated. In general, the mass spectrometry unit 400 includes an inlet sample port 402 configured to an ionizing chamber 404 that has been evacuated with a vacuum pump (not shown). The ionizing chamber 404 includes an ion source 406 in fluid communication with the sample port 402. The ion source 406 is used to ionize the sample to produce precursor ions. An ion guide 408 is configured within the ionizing chamber 404 to transport the precursor ions from the ion source 406 to a mass analyzer unit 409. In general, the mass analyzer unit 409 is used to separate a fraction of the ions based on a mass-to-charge ratio. In some aspects, the mass analyzer 409 may also be configured to dissociate a portion of the precursor ions into fragment ions. The fraction of ions that passes through the mass analyzer unit 409 may then be transferred to a detector 420. The fraction of ions may be oriented to hit the detector to produce detection signals, as is the case for sector or time-of-flight instruments. While, in some aspects, the fraction of ions may pass near the detection plates to produce the detection signals, as is the case in Fourier transform ion cyclotron resonance mass spectrometry (FT ICR). The detection signals may then be transformed into chromatograph or mass spectra using a data processor 428 and a controller 422.

Suitable samples for the mass spectrometry unit 400 system include macromolecules comprising monomer subunits or small molecules. In one non-limiting example, the sample includes a glycan comprising monosaccharide monomer subunits. A suitable mass analyzer unit 409 may include a first quadrupole mass filter 410, a collision cell 412, and a second quadrupole mass filter 418. In general, the first and second quadrupole mass filters 410, 418 include several rod electrodes which may be configured to receive a predetermined amount of voltage that causes a fraction of ions to separate when passing through the quadrupole mass filters 410, 418. The separation is determined by the mass-to-charge ratio (m/z) of the ions. In general, the collision cell 412 includes a multipole ion guide 414 and a gas supply unit 416 that are configured to impart a collision between incoming precursor ions from the first mass filter 410, and an inert gas to induce further dissociation or fractionation of the precursor ions to produce fragment ions. The multipole ion guide 414 is also configured to receive a predetermined amount of voltage for focusing and controlling the position of the ions within the collision cell 412. The gas supply unit 416 is configured to deliver an inert gas (e.g., nitrogen, helium) into the collision cell 412.

The mass spectrometry unit 400 also includes a controller 422 that may include a display 424, one or more input devices 426 (e.g., a keyboard, a mouse), and a data processor 428. The data processor 428 may include a commercially available programmable machine running on a commercially available operating system. The data processor 428 is configured to be in electrical communication with the detector 420 and the controller 422. The controller 422 provides an operator interface that facilitates entering input parameters into the mass spectrometry unit 400. The controller 422 may be configured to be in electrical communication with several power units, including, for example, a first quadrupole power unit 430, a multiple ion guide power unit 32, and a second quadrupole power unit 434. The first quadrupole power unit 430 is further in electrical communication with the first quadrupole mass filter 410. Similarly, the multipole ion guide power unit 432 and the second quadrupole power unit 434 are in electrical communication with the multipole ion guide 414 and the second quadrupole mass filter 418, respectively. The controller 422 may control the data processor 428, one or more input devices 426, and display 424 to implement similar or the same methods described with reference to FIGS. 2-3.

Under the command of the controller 422, predetermined amounts of voltage may be applied to the first quadrupole power unit 430, the multiple ion guide power unit 432, and the second quadrupole power unit 434. The voltages applied from the first and second quadrupole power unit 430, 434 to the first and second quadrupole mass filters 410 and 418 may comprise radio-frequency voltage added to a DC voltage. The voltage applied from the multiple ion guide power unit 432 to the multiple ion guide 414 may be a radio-frequency voltage. In some aspects, a DC bias voltage is additionally applied to the first and second quadrupole mass filters 410, 418 as well as the multiple ion guide 414.

In operation, a sample is injected into the inlet sample port 402 and is ionized by the ion source 406 to produce precursor ions. The ion guide 408 directs the precursor ions into the first quadrupole mass filter 410. The controller 422 determines the amount of voltage to apply to the first quadrupole mass filter 410, which regulates how many precursor ions are allowed to pass through the first quadrupole mass filter 410 based on a specific mass-to-charge ratio (m/z). A fraction of the precursor ions are subsequently fed into the collision cell 412. The controller 422 determines an amount of voltage to apply to the multiple ion guide 414 to focus and position the ions. The controller 422 then regulates an amount of gas to be introduced from the gas supply unit 416 into the collision cell 412. The gas collides with the ions from the first quadrupole mass filter 410 to produce fragment ions.

The precursor and fragment ions are then passed through the second quadrupole power unit 418, where the ions are filtered a second time. To filter the ions, the controller 422 regulates the amount of voltage delivered to the second quadrupole mass filter 418 to again separate a fraction of the precursor and fragment ions based on a mass-to-charge ratio. The fraction of precursor and fragment ions are then directed to the detector 420 where a detection signal corresponding to the number of each incident ions is produced, and the detection signal is subsequently sent to the data processor 428. The detection signal may be generated by contacting the detector 420, or it may be generated by passing near the detector 420.

The data processor 428 may communicate with the controller 422 to execute stored functions that can create chromatographs and mass spectra based on the data produced from the detection signals by digitizing the signal fed from the mass spectrometry unit 400. The data processor may also perform qualitative and quantitative determination processes based on the chromatograph or mass spectra. Chromatograph or mass spectra data may be conveyed back to the controller 422 where they are stored in data base memory cache, from which they may be transferred to the display 424. In other aspects, the computer system 300 may be integrated into the mass spectrometry unit 400.

In some aspects, the mass spectrometry unit 400 may be configured to acquire a mass spectrum of a molecule that includes mass spectrum peaks corresponding to a precursor ion and fragment ions. The term precursor ion may be produced by using the ion source 306, and the fragment ions may be produced in the collision cell 412 (e.g., O-ion fragments). For example, the macromolecule may pass through the ion source 406 to acquire a charge, or partially fragment and acquire a charge to produce a precursor ion. The precursor ion may then be passed through the collision cell 412 to further dissociate and fragment the precursor ions to produce fragment ions. The mass spectrometry unit 400 may be configured to implement the same or similar methods as described in FIGS. 2-3.

It is to be appreciated that alternative mass spectrometry units may be used in accordance with the present disclosure. In general, any mass spectrometry unit capable of ionizing chemical species and separating them based on their mass-to-charge ratio may be used in accordance with the present disclosure. Suitable examples may include AMS, GC-MS, LC-MS, ICP-MS, IRMS, MALDI-TOF, SELDI-TOF, Tandem MS, TIMS, SSMS, and similar mass spectrometry instruments.

### EXAMPLES

The following examples set forth, in detail, ways in which the systems and methods provided herein may be used or implemented, and will enable one of skill in the art to more readily understand the principles thereof. The following examples are presented by way of illustration and are not meant to be limiting in any way.

### Example 1

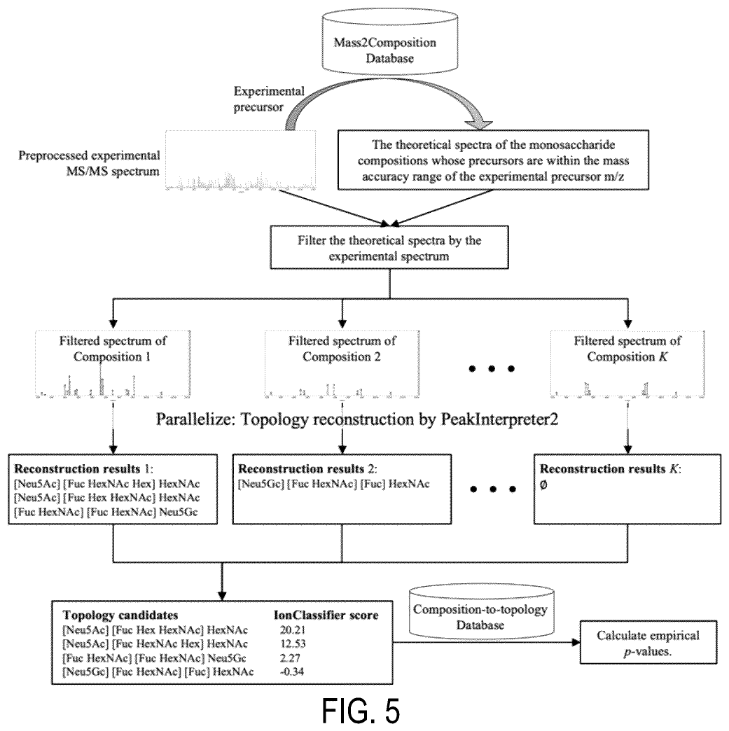

FIG. 5 is a schematic flowchart that illustrates a non-limiting example method of determining a topology for a biomolecule in accordance with some aspects of the present disclosure. As shown in FIG. 5, given an experimental MS/MS spectrum, the method which is also referred to as “GlycoDeNovo2,” first preprocesses the MS/MS spectrum, and then uses the protonated precursor mass to retrieve at least a portion or all matched monosaccharide compositions and their theoretical spectra from a precomputed mass-to-composition database DBM2C.

The retrieved theoretical spectra are filtered by the preprocessed experimental spectrum (i.e., the spectrum produced by removal of theoretical peaks that cannot be matched to experimental peaks within the specified mass accuracy). The PeakInterpreter function of GlycoDeNovo was modified to use the retrieved compositions and their filtered theoretical spectra to speed up the topology search. This is advantageous, because using the filtered theoretical spectrum prevents error propagation, especially in computing the complementary peaks. In GlycoDeNovo, a complementary peak is calculated using the experimental precursor peak and a selected experimental peak. Hence, the mass measurement error in both experimental peaks can be accumulated into the computed complementary peak and further propagated in the downstream computations. This can be avoided by using the theoretical mass value of the selected precursors, as their mass measurements are accurate.

The IonClassifier of GlycoDeNovo is used to score the peaks (i.e., the possibility of a peak being a B-/C-ion) in the spectrum. A score is derived for each topology candidate by summing up the scores of its supporting B-/C-ions (peaks). Finally, GlycoDeNovo2 calculates an empirical p-value for the score of each reconstructed candidate. The p-value calculation uses a composition-to-topology database DBC2T, which can be precomputed.

Throughout the rest of Example 1, G is used to indicate the set of all monosaccharide classes being considered and k=|G| to indicate the size of G. Let C=[c₁, c₂, . . . , cₖ] be the monosaccharide composition, where cᵢ is the number of the i-th monosaccharide class in the composition, and the monosaccharide classes are ordered from the lightest to the heaviest. In some aspects, monosaccharides are not distinguished in the same class, as they are not distinguishable by MS/MS. For example, Glucose, Galactose and Mannose are all treated as Hex. Hereafter, monosaccharides are used to indicate “monosaccharide class”.

### Spectrum Preprocessing:

The preprocessing procedure first protonates all peaks in a given MS/MS spectrum. It is common that some glycosidic fragments might not be observed due to secondary fragmentations, or lack of charge carriers. Without those missing peaks, our topology reconstruction algorithm may fail to derive the right candidates. In theory, when a glycan is cleaved only once, two complementary ions should appear. Hence, missing peaks can be recovered from their complementary peaks. For example, B-/C-/A-ions can be recovered from Y-/Z-/X-ions, respectively, and vice versa. Since the precursor ion is known, we can calculate the complementary peak of each experimentally observed peak and add a computed peak to the spectrum if it is missing in the original spectrum. Then we iteratively merge peaks that are within 0.001 Dalton starting from the closest pair of peaks.

### Mass-to-Composition Database:

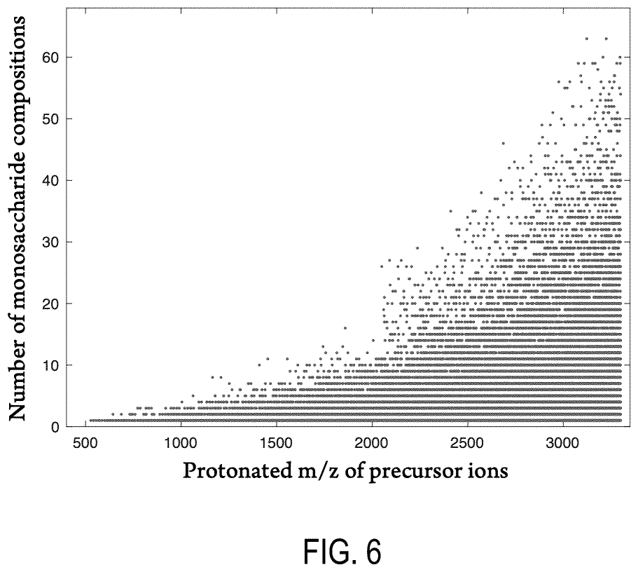

The mass-to-composition database DB_{M2C} is indexed by precursor masses and stores at least a portion or all possible monosaccharide compositions of glycans with precursor masses smaller than a predefined threshold Mmax. DB_{M2C} also stores the theoretical MS/MS spectra corresponding to each monosaccharide composition. Two algorithms, Mass2Composition and Composition2Spectrum, were designed and implemented to create DB_{M2C}. Mass2Composition (Algorithm 1) efficiently derives a portion or all monosaccharide compositions in a recursive way. It starts from an empty composition and calls itself recursively to expand the composition by adding one monosaccharide each time. FIG. 6 shows that larger masses tend to have more monosaccharide compositions. For each monosaccharide composition and a specified derivatization method, Composition2Spectrum (Algorithm 2) calculates the theoretical spectra of a monosaccharide composition as the union of all protonated B-/C-/Y-/Z-ions produced from all possible glycans satisfying the composition constraint.

### Composition-Constrained PeakInterpreter:

The PeakInterpreter algorithm of GlycoDeNovo builds an interpretation-graph that specifies how to interpret each peak using the sub-topology reconstructed for other lighter peaks. By back-tracing the interpretation-graph, we are able to obtain all topology candidates. PeakInterpreter maintains a pool of candidates, each of which serves as a potential building block for interpretation of a heavier peak. PeakInterpreter starts from the lightest peak and tries to interpret every peak as a B-ion, C-ion or the precursor ion by searching for all allowable combinations of building blocks in the candidate pool that can be appended to a monosaccharide to derive a candidate set matching a heavier peak. The runtime of PeakInterpreter depends on the number of peaks to be interpreted and can increase significantly as the peak number increases. In the present disclosure, PeakInterpreter was improved to derive PeakInterpreter2 (Algorithm 3) that utilizes the monosaccharide composition constraint to dramatically reduce the search space for the following two reasons. First, PeakInterpreter2 only needs to interpret the experimental peaks that can be matched to those theoretically allowed by the composition constraint, which dramatically reduces the number of peaks to be interpreted. Second, PeakInterpreter2 does not need to examine the topologies that break the composition constraint.

### Composition-to-Topology Database:

The composition-to-topology database DB_{C2T} allows one to retrieve a plurality or all topologies using a monosaccharide composition query. DB_{C2T} organizes topologies and their sub-topologies into topology sets and topology super sets. A topology super set contains all topologies (or sub-topologies) of the same monosaccharide composition, which are organized in topology sets. A topology set contains topologies (or sub-topologies) that have the same monosaccharide composition, are rooted at the same monosaccharide, and share the same branching pattern at its root. A branching pattern specifies the number of branches of all topologies (or sub-topologies) in this topology set and the monosaccharide composition of each branch (i.e., each branch contains a set of sub-topologies in a topology super set). The topology sets and topology super sets are stored in two cross-referred databases, DB_{C2TS} and DB_{C2TSS}, respectively. DBczTs and DBczTss together effectively organize all topologies and sub-topologies in a directed acyclic graph (DAG), which is similar to the interpretation-graph. Each node in this DAG is either a topology set or a topology super set. A comprehensive DB_{C2T} can be pre-computed by traversing this DAG and be used later in calculating the p-value of a topology candidate. It is also indexed by the masses of topologies and stores the theoretical spectrum of each topology. This process may be time consuming, but it fortunately only needs to be run once. For very large glycans, the number of possible topologies can be too large to pre-compute and store offline. For the purpose of computing empirical p-values, we can instead sample the DAG to obtain the desired number of topologies.

The construction of DB_{C2TS} and DB_{C2TSS} utilizes two algorithms, Composition2TSS (Algorithm 4) and CreateRootedTSS (Algorithm 5). Composition2TSS takes a monosaccharide composition C=[c₁, c₂, . . . , cₖ] as input and recursively reconstructs and saves a plurality or all possible typologies (or sub-topologies) satisfying this composition. The algorithm iterates through available monosaccharides in C. Each time, it picks a monosaccharide, say mᵢ, as a root, and then calls the algorithm CreateRootedTSS (Algorithm 4) with the remaining composition to create all topologies (or sub-topologies) rooted at mi.

### Calculate Empirical P-Value of Topology Candidate:

After reconstructing the topology candidates using PeakInterpreter2, the IonClassifier of GlycoDeNovo is used to score each peak in the given experimental spectrum. A score is derived for each topology candidate by summing up the IonClassifier scores of its supporting peaks. Note that each peak is given a score (the probability of being a B-/C-ion) by IonClassifier. To avoid double counting, Y-/Z-ions are not counted as they are complementary to B-/C-ions. We can rank the topology candidates by their scores, which however do not indicate their statistical significance. Hence, we need to obtain the corresponding p-values to assess the likelihood of obtaining such a topology by random. GlycoDeNovo2 takes an empirical approach to achieve this. First, it samples with replacement a large number of topologies (currently set as up to the max of 10000 or 10% of the total population), whose masses are within the mass accuracy of the experimental precursor mass, from the pre-computed composition-to-topology database DB_{C2T}. The theoretical spectrum for each sampled topology is matched against the experimental spectrum, and the IonClassifier scores of the matched peaks are summed up to derive a score of the sampled topology. The scores of all sampled topologies form an empirical distribution that can be used to derive a p-value for the score of a topology candidate reconstructed by PeakInterpreter2.

### Experimental Results:

To test GlycoDeNovo2, 128 electronic excitation dissociation (EED) MS/MS spectra were used with their precursor mass values ranging between 668.35 Da to 3188.59 Da. Twenty-nine of these spectra were produced by synthetic or purified glycan

standards (Table 1) [22], and the rest were generated by LC-MS/MS analyses of glycans released from glycoprotein standards ribonuclease B and bovine submaxillary mucin, and glycoproteins in human serum, and derivatized as indicated in Table 2. A porous graphitic carbon (PGC) column was used for online LC separation because it achieves the highest performance in resolving isomeric structures. EED MS/MS spectra were recorded on a 12-T solariX hybrid Qh-Fourier-transform ion cyclotron resonance (FTICR) mass spectrometer (Bruker Daltonics, Bremen, Germany).

Each spectrum was acquired with a 0.5-s transient, resulting in a typical mass resolving power of around 191,000 at m/z 400. All spectra were manually interpreted based on our current knowledge of the EED fragmentation process and the glycan biosynthetic pathways. The peak assignment mass accuracy is typically 1 ppm or better for spectra acquired by direct infusion, and 2 ppm or better for spectra acquired by LC-MS/MS. All 128 spectra were used in comparing the speeds of GlycoDeNovo and GlycoDeNovo2, but only those produced by glycan standards with known structures were used in demonstrating the p-value calculation function of GlycoDeNovo2.

### Runtime Comparison:

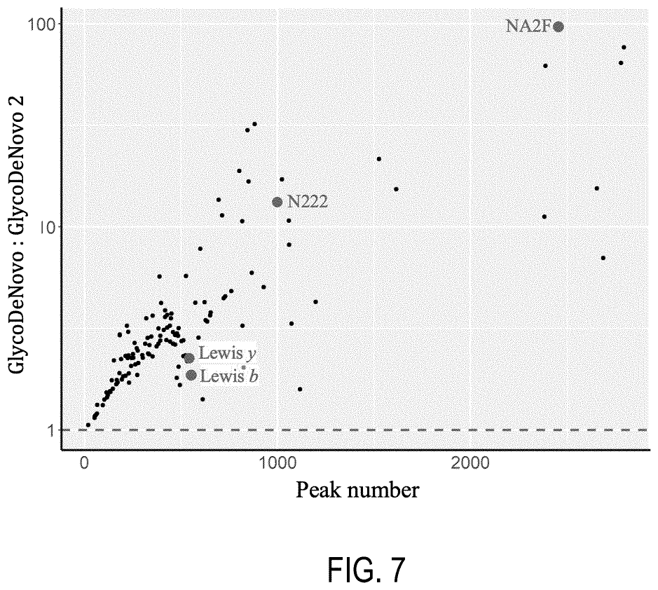

We implemented GlycoDeNovo2 based on GlycoDeNovo by adding the monosaccharide composition constraint and parallel computing. FIG. 7 compares the efficiency and scalability of GlycoDeNovo2 and GlycoDeNovo. They were both run on computers of the same setting (Intel® Core™ 17-9750H CPU @ 2.60 GHz, 256.0 GB RAM) for a fair comparison. Each reconstruction thread only uses one CPU core. To deal with uncontrollable system fluctuations, we ran both algorithms 10 times on each MS/MS spectrum and calculated the mean of the ratios between their runtimes. In all cases, GlycoDeNovo2 runs significantly faster than GlycoDeNovo, and this speed advantage is more pronounced for larger glycans that tend to generate a higher number of peaks in their tandem mass spectra. For example, on small glycans (e.g. Lewis b and Lewis y), GlycoDeNovo2 runs ˜5 faster than GlycoDeNovo. The speed advantage of GlycoDeNovo2 is more pronounced on larger glycans, which tend to produce more peaks in their spectra. For example, GlycoDeNovo2 runs ˜10 times faster on N222 and ˜100 times faster on NA2F. With this improvement in running speed, it is possible to reconstruct topologies from MS/MS data in real-time, even for large glycans. This ability is important to intelligent selection of MS² fragments for MS³ analysis following on-line LC separation.

### Time Complexity Analysis:

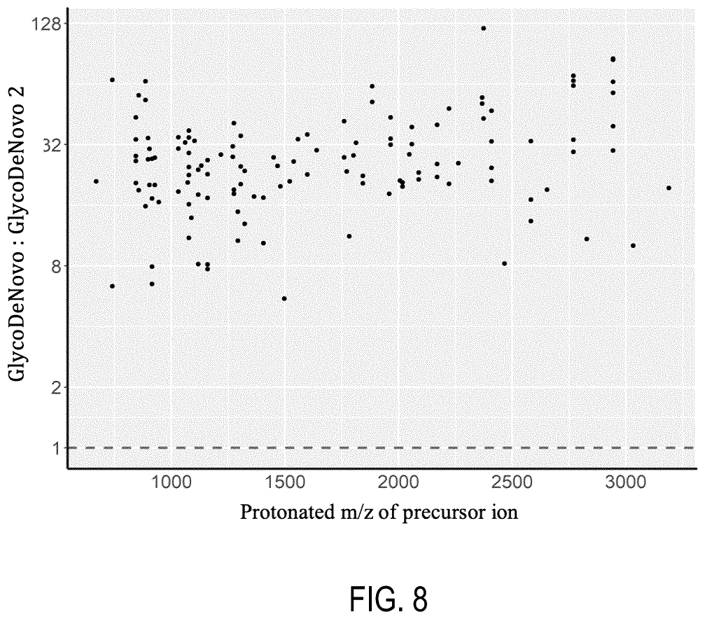

The time complexity of GlycoDeNovo PeakInterpreter is o(|G|×N^{H+1}), where G is the set of the allowed monosaccharide classes, N is the number of peaks in the MS/MS spectrum being considered, and H (1≤H≤4) is the maximum branching number allowed in glycans and can be adjusted by users to match with their data. The number of peaks is a key base factor affecting the speed. As glycan structures become more complicated, the number of MS/MS peaks in general increases, which results in an exponential growth in running time. GlycoDeNovo2 utilizes the composition constraint to significantly reduce the number of peaks that need to be considered (FIG. 8). In our experiments, GlycoDenovo2 on average only uses ˜4.5% of peaks considered by GlycoDeNovo. Taking the spectrum of Sialyl Lewis a (SLA) as an example, GlycoDeNovo needs to interpret 459 peaks. GlycoDeNovo2 first retrieves three monosaccharide compositions: [2 Fuc, 1 HexNAc, 1 Neu5Gc], [1 Fuc, 1 Hex. 1 HexNAc, 1 Neu5Ac] and [2 Xyl, 1 Fuc, 2 HexNAc], where each digit indicates the number of the following monosaccharide contained in a legal topology candidate. The corresponding three filtered spectra have only 15, 24, and 20 peaks, respectively, which are substantially lower than the number of peaks in the original spectrum. As the result, GlycoDeNovo2 runs 6.5 faster than GlycoDeNovo in this case.

### Empirical P-Values of Reconstructed Topologies:

Like GlycoDeNovo, GlycoDeNovo2 is able to correctly reconstruct the topologies of glycans in Table 1. In addition, GlycoDeNovo2 calculates the statistical significance of the topology candidates. Table 2 lists the empirical p-values of the correct topology candidates for the glycans in Table 1, and clearly indicates the correct topology candidates for those glycans are statistically significant.

TABLE 1
Glycan standards used in this Example.

| Short | | Structure (CFG with linkage |
|---|---|---|
| Name | Formula | placement notation) |
| SLa | [Neu5Ac(α2-3) Gal(β1-3)] [Fuc(α1-4)] GlcNAc | 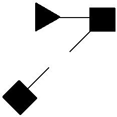 |
| SLx | [Neu5Ac(α2-3) Gal(β1-4)] [Fuc(α1-3)] GlcNAc | 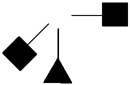 |
| Lewis b | [Fuc(α1-2) Gal(β1-3)] [Fuc(α1-4)] GlcNAc | 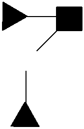 |
| Lewis y | [Fuc(α1-2) Gal(β1-4)] [Fuc(α1-3)] GlcNAc | 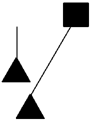 |
| LNT | Gal(β1-3) GlcNAc(β1-3) Gal(β1-4) Glc | 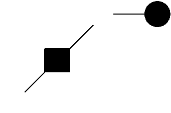 |
| LNnT | Gal(β1-4) GlcNAc(β1-3) Gal(αβ1-4) Glc | 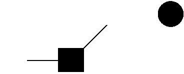 |
| LNFP I | Fuc(α1-2) Gal(β1-3) GlcNAc(β1-3) Gal(β1-4) Glc | 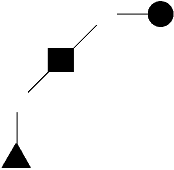 |
| LNFP II | [Gal(β1-3)] [Fuc(α1-4)] GlcNAc(β1-3) Gal(β1-4) Glc | 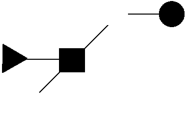 |
| LNFP III | [Gal(β1-4)] [Fuc(α1-3)] GlcNAc(β1-3) Gal(β1-4) Glc | 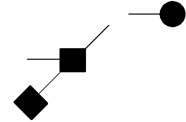 |
| CelHex | Glc(β1-4) Glc(β1-4) Glc(β1-4) Glc(β1-4) Glc(β1-4) Glc | 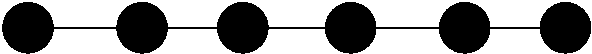 |
| MalHex | Glc(α1-4) Glc(α1-4) Glc(α1-4) Glc(α1-4) Glc(α1-4) Glc | 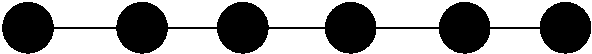 |
| N002 | [Neu5Ac(α2-3) Gal(β1-4) GlcNAc(β1-2) Man(α1-3)] [Neu5Ac(α2-3) Gal(β1-4) GlcNAc(β1-2) Man(α1-6)] Man(β1-4) GlcNAc(β1-4) GlcNAc | 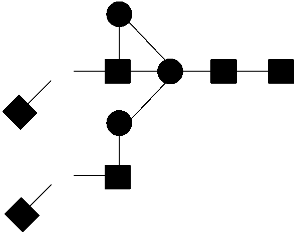 |
| N003 | [Neu5Ac(α2-6) Gal(β1-4) GlcNAc(β1-2) Man(α1-3)] [Neu5Ac(α2-6) Gal(β1-4) GlcNAc(β1-2) Man(α1-6)] Man(β1-4) GlcNAc(β1-4) GlcNAc | 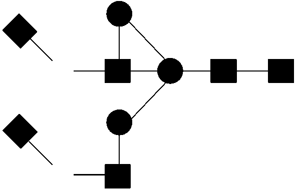 |
| N012 | [Neu5Ac(α2-3) Gal(β1-4) GlcNAc(β1-2) Man(α1-3)] [[Man(α1-3)] [Man(α1-6)] Man(α1-6)] Man(β1-4) GlcNAc(β1-4) GlcNAc | 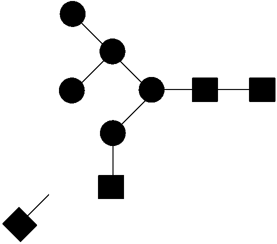 |
| N013 | [Neu5Ac(α2-6) Gal(β1-4) GlcNAc(β1-2) Man(α1-3)] [[Man(α1-3)] [Man(α1-6)] Man(α1-6)] Man(β1-4) GlcNAc(β1-4) GlcNAc | 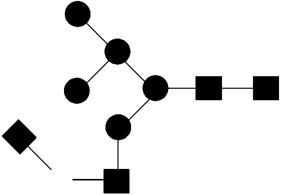 |
| N222 | [Neu5Ac(α2-3) Gal(β1-4) GlcNAc(β1-2) Man(α1-6)] [Gal(β1-4) GlcNAc(β1-2) Man(α1-3)] Man(β1-4) GlcNAc(β1-4) GlcNAc | 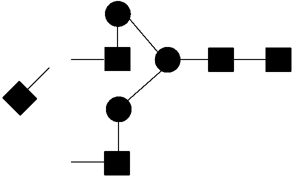 |
| N223 | [Neu5Ac(α2-6) Gal(β1-4) GlcNAc(β1-2) Man(α1-6)] [Gal(β1-4) GlcNAc(β1-2) Man(α1-3)] Man(β1-4) GlcNAc(β1-4) GlcNAc | 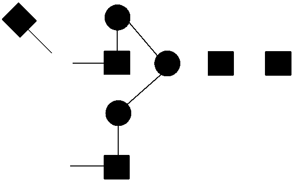 |
| N233 | [Neu5Ac(α2-3) Gal(β1-4) GlcNAc(β1-2) Man(α1-3)] [Neu5Ac(α2-6) Gal(β1-4) GlcNAc(β1-2) Man(α1-6)] Man(β1-4) GlcNAc(β1-4) GlcNAc | 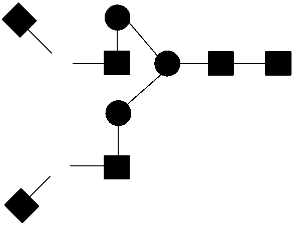 |
| NA2F | [Gal(β1-4) GlcNAc(β1-2) Man(α1-6)] [Gal(β1-4) GlcNAc(β1-2) Man(α1-3)] Man(β1-4) GlcNAc(β1-4) [Fuc(α1-6)] GlcNAc | 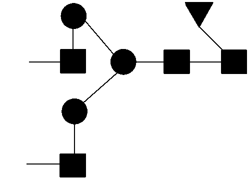 |
| A2F | [Neu5Ac(α2-6) Gal(β1-4) GlcNAc(β1-2) Man(α1-3)] [Neu5Ac(α2-6) Gal(β1-4) GlcNAc(β1-2) Man(α1-6)] Man(β1-4) GlcNAc(β1-4) [Fuc(α1-6)] GlcNAc | 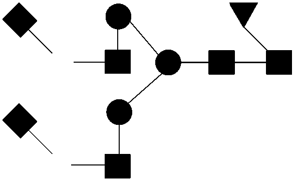 |
| Man9 | [[Man(α1-2) Man(α1-6)] [Man(α1-2) Man(α1-3)] Man(α1-6)] [Man(α1-2) Man(α1-2) Man(α1-3)] Man(β1-4) GlcNAc(β1-4) GlcNAc | 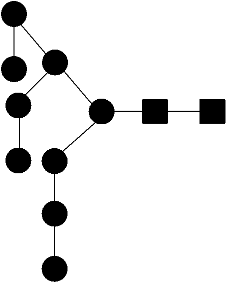 |

TABLE 2
Empirical p-values. All glycans are permethylated. The “REM” column
indicates the type of reducing end modifications (018 = 180-labeled,
D-R = deutero-reduced, Red = reduced). The “#Peaks Used by
GlycoDeNovo” column lists the peak number of each preprocessed spectrum
(i.e., used by PeakInterpreter of GlycoDeNovo). The “#Peaks Used by
GlycoDeNovo2” column lists the peak number in each filtered spectrum
used by PeakInterpreter2 of GlycoDeNovo2. Some have multiple filtered spectra.
For example, N002 has 8 filtered spectra. The “#Candidates” column
lists the number of the reconstructed topology candidates. The “p-value”
column lists the empirical p-values of the correcttopologies.

| | | | #Peaks Used by | #Peaks Used by | | |
|---|---|---|---|---|---|---|
| Glycan | REM | Metal | GlycoDeNovo | GlycoDeNovo2 | #Candidates | p-value |
| Lewis b | O18 | Cs | 329 | 10 | 3 | 0.03571 |
| Lewis b | O18 | Na | 216 | 11 | 4 | 0.03571 |
| Lewis y | O18 | Cs | 461 | 11 | 4 | 0.03571 |
| Lewis y | O18 | Na | 283 | 11 | 3 | 0.03571 |
| LNFP I | O18 | Cs | 469 | 12 | 16 | 0.01333 |
| LNFP I | O18 | Na | 516 | 8 | 13 | 0.01333 |
| LNFP II | O18 | Cs | 390 | 10 | 16 | 0.01333 |
| LNFP II | O18 | Na | 534 | 12 | 21 | 0.01333 |
| LNFP III | O18 | Cs | 471 | 10 | 16 | 0.01333 |
| LNFP III | O18 | Na | 477 | 8 | 17 | 0.01333 |
| LNFP II | D-R | Na | 546 | 17 | 13 | 0.01333 |
| NA2F | O18 | Na | 2389 | 23 | 101 | <10-5 |
| Man9 | O18 | Na | 2532 | 26/42/42/39 | 468 | <10-5 |
| A2F | Red | Na | 2646 | 56/105/126/78/111/ | 1012216 | <10-5 |
| | | | | 95/153/102 | | |
| A2F | D-R | Na | 914 | 28/34/17/23/20/40/ | 37 | <10-5 |
| | | | | 29 | | |
| N002 | D-R | Na | 2320 | 28/33/63/59/46/55/ | 157478 | <10-5 |
| | | | | 95/52 | | |
| N003 | D-R | Na | 1571 | 19/23/47/44/30/40/ | 1056 | <10-5 |
| | | | | 65/32 | | |
| N012 | D-F | Na | 2683 | 32/57/46 | 5001 | <10-5 |
| N013 | D-R | Na | 2544 | 31/45/42 | 3767 | <10-5 |
| N222 | D-R | Na | 953 | 20/34/37 | 51 | <10-5 |
| N223 | D-R | Na | 2674 | 36/54/61 | 14963 | <10-5 |
| N233 | D-R | Na | 2326 | 27/28/60/65/47/49/ | 2557 | <10-5 |
| | | | | 93/47 | | |
| Lewis b | None | Na | 218 | 13 | 4 | 0.03571 |
| LNT | None | Na | 317 | 7 | 5 | 0.1 |
| LNnT | None | Na | 270 | 9 | 5 | 0.1 |
| SLa | None | Na | 459 | 11/17/15 | 14 | 0.00521 |
| SLx | None | Na | 333 | 13/19/17 | 22 | 0.00521 |
| CelHex | None | Na | 412 | 11 | 11 | 0.09091 |
| MalHex | None | Na | 468 | 11 | 22 | 0.09091 |

### Conclusions:

GlycoDeNovo2 is a fast algorithm for de novo reconstruction of glycan topologies from MS/MS data. It offers a functionality to calculate the p-values of the reconstructed topologies. It allows determination of the monosaccharide compositions for glycans satisfying any given precursor mass, within defined mass measurement accuracy limits, which can then be used to constrain the search space of potential topologies. The mapping from masses to monosaccharide compositions can be precomputed. A theoretical spectrum can be pre-computed for each monosaccharide composition to include the theoretical glycosidic fragments of all topology candidates satisfying the monosaccharide composition constraint. Given an experimental MS/MS spectrum, GlycoDeNovo2 retrieves a plurality or all monosaccharide compositions and their theoretical spectra, which are within the mass accuracy of the experimental precursor mass. The retrieved theoretical spectra are then filtered by the experimental spectrum before being used for reconstructing topology candidates. The number of peaks in such a filtered theoretical spectrum is substantially smaller than that in the experimental spectrum. Hence, it takes considerably shorter time to reconstruct topologies from a filtered theoretical spectrum.

In addition, the reconstruction process for each monosaccharide composition can run independently, i.e., GlycoDeNovo2 can parallelize the reconstruction processes for all monosaccharide compositions. Experimental results show that GlycoDeNovo2 runs significantly faster than its predecessor GlycoDeNovo. Existing topology reconstruction algorithms assign a numerical score to each topology candidate. However, the statistical significance of such a score is unknown. GlycoDeNovo2 deploys a procedure to calculate the empirical p-values of a reconstructed topology candidate. In our experiments, a set of standard glycans, whose structures are known, were used to demonstrate that GlycoDeNovo2 can reconstruct the correct topologies with significant p-values.

The present disclosure has described one or more preferred embodiments, and it should be appreciated that many equivalents, alternatives, variations, and modifications, aside from those expressly stated, are possible and within the scope of the invention.

### Experimental Methods

### Materials

Sialyl Lewis a, sialyl Lewis x, Lewis b, Lewis y, lacto-N-tetraose (LNT), and lacto-N-neotetraose (LNnT) were acquired from Dextra Laboratories (Reading, UK). Lacto-N-fucopentaose (LNFP) I, II, and III were purchased1 from V-LABS, Inc. (Covington, LA, USA). Cellohexaose (CelHex), maltohexaose (MalHex), A2F, and NA2F glycans were acquired from Carbosynth Ltd. (Berkshire, UK). Synthetic N-linked glycan standards (N002 to N233) were obtained from Chemily Glycoscience (Atlanta, GA, USA). PNGase F was purchased from New England BioLabs (Ipswich, MA). Man9 N-glycan, human blood serum, bovine submaxillary mucin, dithiothreitol (DTT), H₂¹⁸O (97%) water, 2-aminopyridine (2-AA), acetic acid, dimethyl sulfoxide (DMSO), sodium hydroxide, methyl iodide, ammonium bicarbonate (ABC), sodium borodeuteride, and cesium acetate were purchased from Sigma-Aldrich (St. Louis, MO, USA). HPLC grade water, acetonitrile (ACN), chloroform, isopropyl alcohol (IPA), and formic acid (FA) were acquired from Fisher Scientific (Pittsburgh, PA). C18 Sep-Pak cartridges were obtained from Waters (Milford, MA). HyperSep Hypercarb SPE cartridges were purchased from Thermo Fisher Scientific (Waltham, MA).

### Glycan Releases

N-linked glycans were released from human blood serum using PNGase F. Briefly, 10 μL of human serum was diluted in 40 μL of water, then centrifuged at 13,000 rpm for 20 min. The supernatant was transferred to a new vial, to which 146 μL of 100 mM ABC buffer and 2 μL of 200 mM DTT were added. The mixture was incubated at 60° C. for 40 min. followed by addition of a 2-μL aliquot of the PNGase F solution, and incubation at 37° C. for 16 hr.

O-linked glycans were released from bovine submaxillary mucin via reductive alkaline β-elimination. Briefly, 1 mg of mucin powder was dissolved in 400 μL aqueous solution of 50 mM NaOH and 50 mM NaBD₄, and incubated at 45° C. for 16 hr. The reaction was terminated by dropwise addition of 10% acetic acid until bubbling ceased.

Released N- and O-linked glycans were purified using C18 Sep-Pak cartridges. The mixture was passed three times through a C18 Sep-Pak cartridge, and then the cartridge was washed three times with 100 μL 5% ACN. All eluents from the C18 cartridge were combined and dried in a SpeedVac concentrator (Thermo Fisher Scientific).

### Reducing-End Modifications

For reducing-end ¹⁸O-isotope labeling. 5 μg of dry native glycan was dissolved in 20 μL of H₂¹⁸O containing 2 μL of the catalyst solution (2.7 mg/mL 2-AA in anhydrous methanol) and 1 μL of acetic acid. The reaction was allowed to proceed at 65° C. for 16 hr. Solvent was removed by a SpeedVac concentrator before permethylation. For deutero-reduction, approximately 10 μg of each dried glycan standard was dissolved in 200 μL of 0.2 M NH₄OH/0.5 M NaBD₄ aqueous solution and incubated at room temperature for 2 hours while mixing. The reaction was stopped by dropwise addition of 10% acetic acid until bubbling ceased. The reaction mixture was dried in a centrifugal evaporator, and excess borates were removed by repeated resuspension and drying of the samples in methanol.

### Permethylation

Permethylation was performed according to the method described by Ciucanu and Kerek with slight modifications. Briefly, dried glycan powders were resuspended in 100 uL of NaOH/DMSO mixture and vortexed for 1 hr at room temperature, followed by addition of 50 μL methyl iodide. The reaction was allowed to proceed for another hour at room temperature in the dark. Another 100 μL of NaOH/DMSO and 50 μL of methyl iodide were added to the reaction mixture, followed by gentle vortexing at room temperature for 1 hr. This process was repeated three times to ensure complete methylation before the reaction was quenched by addition of 200 μL of chloroform and 200 μL of water. Excess salt was removed by washing with 400 μL of water several times until neutral pH was reached. Permethylated glycans were extracted from the organic layer, desalted using a C18 spin column, and dried in a SpeedVac system.

### Off-Line Mass Spectrometry Analysis

Each permethylated glycan standard was dissolved to a concentration of 2-5 μM in 50/50 (v/v) methanol/water solution, with addition of 20-50 μM of sodium hydroxide or cesium acetate to promote formation of metal adducts. For off-line electronic excitation dissociation (EED) analysis, each glycan sample was loaded onto a pulled glass capillary tip with a 1-um orifice diameter and direct infused into a 12-T solariX hybrid Qh-Fourier transform ion cyclotron resonance (FTICR) mass spectrometer (Bruker Daltonics, Bremen, Germany). Sodiated or cesiated precursor ions were selected by the front-end quadrupole mass filter, accumulated in the collision cell, and fragmented in the ICR cell by electron irradiation time of up to 1 s. The cathode bias was set at −14 V and the ECD lens voltage at −13.95 V. Each transient was recorded for 0.55 s, and up to 40 transients were summed for improved S N ratio.

### On-Line LC-MS/MS Analysis

On-line liquid chromatography separation was carried out on a Waters nanoACQUITY UPLC system (Milford, MA), equipped with a nanoACQUITY UPLC 2G-VMTrap column (5 μm, Symmetry C18, 180 μm ID×20 mm), and a Hypercarb nanoPGC analytical column (3 μm. 75 μm ID×100 mm). The column temperature was kept at 60° C. for optimal chromatographic resolution. Mobile phase A consisted of 98.9% water, 1% ACN, 0.1% formic acid, and mobile phase B consisted of 49.9% ACN, 50% IPA, and 0.1% formic acid. Each injection contained glycans released from approximately 0.2 μL of serum. On-line desalting was carried out by passing sample through the trapping column with 10% B at a flow rate of 4 μL/min for 2 min. The analytical gradient started at 35% B for 5 min, followed by a linear ramp to 95% B over the next 60 min.

Eluted glycans were introduced into the FTICR mass spectrometer via a CaptiveSpray nanoESI source. Auto MS/MS was performed with alternating MS and MS/MS scans. An inclusion list was used without dynamic exclusion to allow the sodiated precursors to be repeatedly selected for fragmentation. Typical precursor ion accumulation time was 0.5 s for MS scans and 1-3 s for MS/MS scans. On-line nanoLC-EED MS/MS analysis was performed with the cathode bias set at 18 V, and an electron irradiation time of 0.5 s. A 0.5-s transient was recorded for each mass spectrum.

### A Reconstruction Example

Here we use an example, Sialyl Lewis a (SLa) [NeuAc (a2-3) Gal (b1-3)] [Fuc (a1-4)] GlcNAc (b1-0), to demonstrate the topology reconstruction flow of GlycoDeNovo2. Using the protonated precursor of SLa (1031.537946 mz) and the mass accuracy 5ppm, GlycoDeNovo2 retrieves 3 possible monosaccharide compositions from DB_{M2C}: [2 Fuc, 1 HexNAc, 1 Neu5Gc], [1 Fuc, 1 Hex, 1 HexNAc, 1 Neu5Ac] and [2 Xyl, 1 Fuc, 2 HexNAc]. The first monosaccharide composition [2 Fuc, 1 HexNAc, 1 Neu5Gc] constrains the search space of PeakInterpreter2 to 11 peaks, and the corresponding reconstruction results are shown below.

```
# Reconstruction results of composition: [2 Fuc, 1 HexNAc, 1 Neu5Gc]
@ Peak 1 mass 189.112135
  ** B: Fuc
@ Peak 2 mass 207.122700
  ** C: Fuc
@ Peak 3 mass 424.217723
  ** C: Neu5Gc
@ Peak 4 mass 434.238458
  ** B: Fuc HexNAc
@ Peak 5 mass 452.249023
  ** C: Fuc HexNAc
@ Peak 6 mass 580.296367
  ** B: Fuc Neu5Gc
@ Peak 7 mass 598.306932
  ** C: Fuc Neu5Gc
@ Peak 8 mass 608.327667
  ** B: [Fuc] [Fuc] HexNAc
@ Peak 11 mass 1031.538113 (Precursor)
  ** T: [Fuc HexNAc] [Fuc] Neu5Gc
  ** T: [Fuc Neu5Gc] [Fuc] HexNAc
  ** T: [Fuc] [Fuc] HexNAc Neu5Gc
  ** T: [Neu5Gc] [Fuc] [Fuc] HexNAc
# Note:
A branch is indicated by “[ ... ]”. For example, “[Fuc HexNAc] [Fuc] Neu5Gc” has two branches “[Fuc HexNAc]” and “ [Fuc]”
```

The second monosaccharide composition [1 Fuc, 1 Hex. 1 HexNAc, 1 Neu5Ac] constrains the search space of PeakInterpreter2 to 17 peaks, and the corresponding reconstruction results are shown below.

```
# Composition: [1 Fuc, 1 Hex, 1 HexNAc, 1 Neu5Ac]
@ Peak 1 mass 189.112135
  ** B: Fuc
@ Peak 2 mass 207.122700
  ** C: Fuc
@ Peak 3 mass 237.133265
  ** C: Hex
@ Peak 4 mass 376.196593
  ** B: Neu5Ac
@ Peak 5 mass 394.207158
  ** C: Neu5Ac
@ Peak 6 mass 434.238458
  ** B: Fuc HexNAc
@ Peak 7 mass 452.249023
  ** C: Fuc HexNAc
@ Peak 8 mass 482.259588
  ** C: Hex HexNAc
@ Peak 9 mass 550.285802
  ** B: Fuc Neu5Ac
@ Peak 10 mass 580.296367
  ** B: Hex Neu5Ac
  ** B: Neu5Ac Hex
@ Peak 11 mass 598.306932
  ** C: Hex Neu5Ac
  ** C: Neu5Ac Hex
@ Peak 12 mass 638.338232
  ** B: Fuc HexNAc Hex
  ** B: [Hex] [Fuc] HexNAc
@ Peak 13 mass 656.348796
  ** C: Fuc HexNAc Hex
  ** C: [Hex] [Fuc] HexNAc
@ Peak 14 mass 795.412125
  ** B: Fuc HexNAc Neu5Ac
  ** B: Fuc Neu5Ac HexNAc
  ** B: [Neu5Ac] [Fuc] HexNAc
@ Peak 17 mass 1031.538113
  ** T: Fuc HexNAc Hex Neu5Ac
  ** T: [Neu5Ac Hex] [Fuc] HexNAc
  ** T: [Neu5Ac] [Fuc HexNAc] Hex
  ** T: Fuc HexNAc Neu5Ac Hex
  ** T: [Hex Neu5Ac] [Fuc] HexNAc
  ** T: [Hex] [Fuc HexNAc] Neu5Ac
  ** T: [Hex] [Fuc] HexNAc Neu5Ac
  ** T: [Neu5Ac] [Fuc] HexNAc Hex
  ** T: [Neu5Ac] [Hex] [Fuc] HexNAc
  ** T: Fuc Neu5Ac HexNAc Hex
  ** T: [Hex HexNAc] [Fuc] Neu5Ac
  ** T: [Hex] [Fuc Neu5Ac] HexNAc
# Note:
A branch is indicated by “[ ... ]”. For example, “[Neu5Ac Hex] [Fuc] HexNAc” has two branches “[Neu5Ac Hex]” and “ [Fuc]”
```

The third monosaccharide composition [2 Xyl, 1 Fuc, 2 HexNAc] constrains the search space of PeakInterpreter2 to 15 peaks, which yields no reconstruction result.

### Data and Software

A public Github repository (https://github.com/Cyrus9721/GlycoDenovo2) contains the data of the 29 glycan standards (Table 1 in main text) and GlycoDeNovo2 (MATLAB executable components and python components) with running instructions.

### REFERENCES

1. Helenius, A. and M. Aebi, Intracellular functions of N-linked glycans. Science, 2001. 291(5512): p. 2364-2369.
2. Ohtsubo, K. and J. D. Marth, Glycosylation in cellular mechanisms of health and disease. Cell, 2006. 126(5): p. 855-867.
3. Varki, A., Biological roles of glycans. Glycobiology, 2017. 27(1): p. 3-49.
4. Dennis, J. W., M. Granovsky, and C. E. Warren, Glycoprotein glycosylation and cancer progression. Biochimica et Biophysica Acta (BBA)-General Subjects, 1999. 1473(1): p. 21-34.
5. Dube, D. H. and C. R. Bertozzi, Glycans in cancer and inflammation-potential for therapeutics and diagnostics. Nature Reviews Drug Discovery, 2005. 4(6): p. 477-488.
6. Jefferis, R., Glycosylation as a strategy to improve antibody-based therapeutics. Nature Reviews Drug Discovery, 2009. 8(3): p. 226-234.
7. Solá, R. J. and K. Griebenow, Glycosylation of therapeutic proteins. BioDrugs, 2010. 24(1): p. 9-21.
8. Dell, A. and H. R. Morris, Glycoprotein structure determination by mass spectrometry. Science, 2001. 291(5512): p. 2351-6.
9. Zaia, J., Mass spectrometry of oligosaccharides. Mass Spectrometry Reviews, 2004. 23(3): p. 161-227.
10. Domon, B.; Costello, C. E. A systematic nomenclature for carbohydrate fragmentations in FAB-MS/MS spectra of glycoconjugates. Glycoconjugate J. 5, 397-409 (1988).
11. Tseng, K., J. L. Hedrick, and C. B. Lebrilla, Catalog-library approach for the rapid and sensitive structural elucidation of oligosaccharides. Analytical Chemistry, 1999. 71(17): p. 3747-54.
12. Joshi, H. J., et al., Development of a mass fingerprinting tool for automated interpretation of oligosaccharide fragmentation data. Proteomics, 2004. 4(6): p. 1650-64.
13. Lohmann, K. K. and C. W. von der Lieth, GlycoFragment and GlycoSearchMS: web tools to support the interpretation of mass spectra of complex carbohydrates. Nucleic Acids Research, 2004. 32 (Web Server issue): p. W261-6.
14. Cooper, C. A., E. Gasteiger, and N. H. Packer, GlycoMod—a software tool for determining glycosylation compositions from mass spectrometric data. Proteomics, 2001.1(2): p. 340-9.
15. Gaucher, S. P ., J. Morrow, and J. A. Leary, STAT: a saccharide topology analysis tool used in combination with tandem mass spectrometry. Analytical Chemistry, 2000. 72(11): p. 2331-6.
16. Ethier, M., et al., Automated structural assignment of derivatized complex N-linked oligosaccharides from tandem mass spectra. Rapid Communications in Mass Spectrometry, 2002. 16(18): p. 1743-54.
17. Ethier, M., et al., Application of the StrOligo algorithm for the automated structure assignment of complex N-linked glycans from glycoproteins using tandem mass spectrometry. Rapid Communications in Mass Spectrometry, 2003. 17(24): p. 2713-20.
18. Tang, H., Y. Mechref, and M. V. Novotny, Automated interpretation of MS/MS spectra of oligosaccharides. Bioinformatics, 2005. 21 Suppl 1: p. 1431-9.
19. Sun, W., et al., A Novel Algorithm for Glycan de novo Sequencing Using Tandem Mass Spectrometry, in Bioinformatics Research and Applications. 2015, Springer International Publishing: Switzerland. p. 320-330.
20. Dong, L., et al., An Accurate de novo Algorithm for Glycan Topology Determination from Mass Spectra. IEEE/ACM Transactions on Computational Biology and Bioinformatics, 2015. 12(3): p. 568-78.
21. Kumozaki, S., K. Sato, and Y. Sakakibara, A Machine Learning Based Approach to de novo Sequencing of Glycans from Tandem Mass Spectrometry Spectrum. IEEE/ACM Transactions on Computational Biology and Bioinformatics, 2015. 12(6): p. 1267-74.
22. Hong, P., et al., GlycoDeNovo-an Efficient Algorithm for Accurate de novo Glycan Topology Reconstruction from Tandem Mass Spectra. J Am Soc Mass Spectrom, 2017. 28(11): p. 2288-2301.
23. Shan, B., et al., Complexities and algorithms for glycan sequencing using tandem mass spectrometry. Journal of Bioinformatics and Computational Biology, 2008. 6(1): p. 77-91.

1. A method for determining a topology for a molecule, the method comprising:
receiving user-defined composition constraints;
acquiring a mass spectrum of a molecule, the mass spectrum including mass spectrum peaks corresponding to a precursor ion and fragment ions, wherein the precursor ion corresponds to an ionized product of the molecule and the fragment ions correspond to dissociated products of the molecule;
matching mass spectrum peaks in the mass spectrum with one or more theoretical mass spectrum peaks of one or more theoretical spectrum of one or more previously-created molecules;
identifying at least a portion of the fragment ions in the mass spectrum as corresponding to one or more monomer subunit ion of the precursor ion, wherein the one or more monomer subunit ion is identified by appending one or more of the fragment ions to an inferable constituent to produce a topology building block, and storing the topology building block in a candidate pool as corresponding to one or more of the monomer subunit ion if the combined mass of the inferable constituent and one or more of the fragment ions satisfy the user-defined composition constraint; and
reconstructing one or more candidate topology of the precursor ion by combining a plurality of the topology building blocks that satisfy the user-defined composition constraints.

2. The method of claim 1, wherein the reconstructing is performed in parallel for each of the one or more candidate topology of the precursor ion.

3. The method of claim 1, wherein the user-defined composition constraints include a first user-defined mass tolerance and a second user-defined mass tolerance for the precursor ion.

4. The method of claim 3, wherein storing the topology building block in the candidate pool as corresponding to one or more of the monomer subunit ion is performed if the combined mass of the inferable constituent and one or more of the fragment ions satisfy the first user-defined mass tolerance.

5. The method of claim 3, wherein reconstructing one or more candidate topology of the precursor ion is performed by combining the plurality of the topology building blocks that satisfy the second user-defined mass tolerance for the precursor ion.

6. A method for determining a topology for a molecule, the method comprising:
acquiring a mass spectrum of a molecule, the mass spectrum including mass spectrum peaks corresponding to a precursor ion and fragment ions, wherein the precursor ion corresponds to an ionized product of the molecule and the fragment ions correspond to dissociated products of the molecule;
matching mass spectrum peaks in the mass spectrum with theoretical mass spectrum peaks of a theoretical spectrum of the molecule;
producing a filtered mass spectrum of the molecule by removing unmatched mass spectrum peaks from the mass spectrum;
identifying at least a portion of the fragment ions in the filtered mass spectrum as corresponding to one or more monomer subunit ion of the precursor ion, wherein the one or more monomer subunit ion is identified by appending one or more of the fragment ions to an inferable constituent to produce a topology building block, and storing the topology building block in a candidate pool as corresponding to one or more of the monomer subunit ion if the combined mass of the inferable constituent and one or more of the fragment ions satisfy a first user-defined mass tolerance; and
reconstructing one or more candidate topology of the precursor ion by combining a plurality of the topology building blocks that satisfy a second user-defined mass tolerance for the precursor ion.

7. The method of claim 6, wherein the reconstructing is performed in parallel for each of the one or more candidate topology of the precursor ion.

8. The method of claim 6, wherein the theoretical spectrum is pre-computed for each monomer subunit composition to include the fragment ions for each of the one or more candidate topology that satisfy the user-defined mass tolerance for the precursor ion.

9. The method of claim 6, further comprising preprocessing the mass spectrum to identify and add in computed complementary peaks missing from the mass spectrum.

10. The method of claim 6, further comprising producing the theoretical spectrum of the molecule by deriving monomer subunit ions recursively that meet a mass tolerance for the molecule and producing the theoretical spectra of the molecule as a union of all protonated monomer subunit ions.

11. The method of claim 6, wherein the molecule is a glycan, and the inferable constituent comprises a monosaccharide.

12. The method of claim 6, wherein the one or more monomer subunit ion comprises a B ion glycosidic fragment or a Cion glycosidic fragment, and the inferable constituent comprises a monosaccharide, and further includes identifying the portion of fragment ions in the mass spectrum as corresponding to B ion glycosidic fragments or C ion glycosidic fragments by attaching up to four branches to the monosaccharide, and wherein the branches are interpretations of fragment ion peaks that are lighter than the one being interpreted.

13. The method of claim 6, further comprising selecting a topology for the precursor ion by ranking the one or more candidate topology based on a candidate topology score.

14. The method of claim 13, wherein the candidate topology score is based on identifying the probability that the fragment ion corresponds to a B ion glycosidic fragment or a C ion glycosidic fragment.

15. The method of claim 13, further comprising generating an empirical p-value for the candidate topology score of the one or more candidate topology.

16. The method of claim 15, wherein generating the empirical p-value includes sampling theoretical topologies from a pre-computed composition-to-topology database to form an empirical distribution, and using the empirical distribution to generate the empirical p-value of the one or more candidate topology.

17. The method of claim 16, wherein the pre-computed composition-to-topology database includes topology sets and topology super sets of the molecule, wherein topology super sets include all topologies of the molecule and are organized into topology sets, and wherein topology sets include topologies of the molecule that are rooted at the same monomer subunit ion and share the same branching pattern at the root.

18. A mass spectrometry unit comprising:
an inlet port configured to receive a sample that includes a molecule comprising monomer subunits;
an ion source configured to ionize the sample to produce a precursor ion, the precursor ion having a first mass-to-charge ratio;
a mass analyzer configured to dissociate a portion of the precursor ion to produce fragment ions, the mass analyzer configured to separate a fraction of the precursor ion and the fragment ions;
a detector configured to produce detection signals corresponding to the fraction of the precursor ion and the fragment ions;
a controller configured to receive the detection signals, the controller programmed to:
acquire a mass spectrum of the molecule, the mass spectrum including mass spectrum peaks corresponding to a precursor ion and fragment ions, wherein the precursor ion corresponds to an ionized product of the molecule and the fragment ions correspond to dissociated products of the molecule;
match mass spectrum peaks in the mass spectrum with theoretical mass spectrum peaks from a theoretical spectrum of the molecule;
produce a filtered mass spectrum of the molecule by removing unmatched mass spectrum peaks from the mass spectrum;
identify at least a portion of the fragment ions in the filtered mass spectrum as corresponding to one or more monomer subunit ion of the precursor ion, wherein the one or more monomer subunit ion is identified by appending one or more of the fragment ions to an inferable constituent to produce a topology building block, and storing the topology building block in a candidate pool as corresponding to one or more of the monomer subunit ion if the combined mass of the inferable constituent and one or more of the fragment ions satisfy a first user-defined mass tolerance; and
reconstruct one or more candidate topology of the precursor ion by combining a plurality of the topology building blocks that satisfy a second user-defined mass tolerance for the precursor ion.

19. The mass spectrometry unit of claim 18, wherein the controller is further programmed to: preprocess the mass spectrum to identify and add in computed complementary peaks missing from the mass spectrum.

20. The mass spectrometry unit of claim 18, wherein the controller is further programmed to: produce the theoretical spectra of the molecule by deriving monomer subunit ions recursively that meet a mass tolerance for the molecule and producing the theoretical spectra of the molecule as a union of all protonated monomer subunit ions.

21. The mass spectrometry unit of claim 18, wherein the molecule is a glycan, and the inferable constituent comprises a monosaccharide.

22. The mass spectrometry unit of claim 18, wherein the one or more monomer subunit ion comprises a B ion glycosidic fragment or a C ion glycosidic fragment, and the inferable constituent comprises a monosaccharide, and further includes identifying the portion of fragment ions in the mass spectrum as corresponding to B ion glycosidic fragments or Cion glycosidic fragments by attaching up to four branches to the monosaccharide, and wherein the branches are interpretations of fragment ion peaks that are lighter than the one being interpreted.

23. The mass spectrometry unit of claim 18, wherein the controller is further programmed to: select a topology for the precursor ion by ranking the one or more candidate topology based on a candidate topology score.

24. The mass spectrometry unit of claim 23, wherein the candidate topology score is based on identifying the probability that the fragment ions correspond to a Bion glycosidic fragment or a C ion glycosidic fragment.

25. The mass spectrometry unit of claim 23, wherein the controller is further programmed to: generate an empirical p-value for the candidate topology score of the one or more candidate topology.

26. The mass spectrometry unit of claim 25, wherein the controller is further programmed to: generate the empirical p-value by sampling theoretical topologies from a pre-computed composition-to-topology database to form an empirical distribution, and using the empirical distribution to generate the empirical p-value of the one or more candidate topology.

27. The mass spectrometry unit of claim 26, wherein the pre-computed composition-to-topology database includes topology sets and topology super sets of the molecule, wherein topology super sets include all topologies of the molecule and are organized into topology sets, and wherein topology sets include topologies of the molecule that are rooted at the same monomer subunit ion, and share the same branching pattern at the root.
